(19) United States
(12) Patent Application Publication
(10) Pub. No.: US 2020/0154998 A1
(43) Pub. Date: May 21, 2020

(54) Test Method for a Disposable

(71) Applicant: Haag-Streit AG, Koeniz (CH)

(72) Inventors: Jörg BREITENSTEIN, Zollikofen (CH); Frank ZUMKEHR, Zollikofen (CH)

(73) Assignee: Haag-Streit AG, Koeniz (CH)

(21) Appl. No.: 16/689,842

(22) Filed: Nov. 20, 2019

(30) Foreign Application Priority Data
Nov. 21, 2018 (CH) 01436/18

(51) Int. Cl. A61B 3/16; A61B 3/135

(52) U.S. Cl. CPC A61B 3/16; A61B 3/14; A61B 3/135

(57) ABSTRACT

In a method for testing a disposable on a medical examination appliance, more particularly on a tonometer, wherein the medical examination appliance comprises a disposable holding apparatus, on which a disposable is held and, in particular, able to be brought into contact with a patient's eye for an applanation tonometry measurement, and which comprises a detection device, by means of which a physical property of the disposable can be measured, a measured value of the physical property of the disposable is ascertained by means of the detection device.

### TECHNICAL FIELD

The invention relates to a method for testing a disposable on a medical examination appliance, more particularly on a tonometer, wherein the medical examination appliance comprises a disposable holding apparatus, on which a disposable is held and, in particular, able to be brought into contact with a patient's eye for an applanation tonometry measurement, and comprises a detection device, by means of which a physical property of the disposable can be measured.

### PRIOR ART

Medical examination appliances are subject to strict hygiene regulations. This should optimally avoid disease transmissions, infections, etc., both in hospitals and in medical practices. Constituent parts of apparatuses, in particular medical examination appliances, which come into direct contact with the patient represent a high risk of contaminations.

In order to reduce the risk of a contamination, such constituent parts are increasingly embodied as so-called disposables. Here, these are constituent parts, or consumables as accessories for a medical examination appliance, which are typically used only once. By way of example, a disposable can be a protective sleeve for a part of an examination appliance that is brought into contact with the patient during an examination or a treatment. Further, this may relate to a constituent part that itself is required to carry out the measurement or the treatment.

In the case of a tonometer, the constituent part can be a test body that is pressed against the cornea of an eye for applanation purposes. Further, the constituent part can also be a contact glass or part of a contact glass for fundoscopy. However, in the case of an otoscope, too, a disposable that may be embodied as a cap and consequently comprises a transparent base as a viewing window may be provided.

U.S. Pat. No. 4,922,914 (Elizabeth O. Segal et al.) discloses, e.g., a protective sleeve in the form of a disposable for a Goldmann tonometer. Direct contact between the tonometer head and patient can be avoided by way of the protective sleeve for the tonometer head. The protective sleeve is replaced for each patient. The protective sleeve further comprises a removable adhesive film on the contact area for the purposes of protecting the contact area.

The disadvantage of the known medical examination appliances, in particular tonometers, is that, for example, the user might forget to replace the disposable with a new disposable between two examinations. This may allow a transmission of pathogens from the first patient to the second patient. Further, use may be made of disposables that, under certain circumstances, are not suitable for the apparatus, as a result of which the measurement results may be impaired. Hence, overall, the use of disposables is linked to many risks. The known medical examination appliances, in particular tonometers, still take too little amount of the safety of the patient, despite the use of disposables.

### SUMMARY OF THE INVENTION

It is a problem of the invention to develop a method that is part of the technical field specified at the outset, by means of which the use of a medical examination appliance can be designed to be safer.

The solution to the problem is defined by the features of claim 1. According to the invention, a measured value of the physical property of the disposable is ascertained by means of the detection device.

This renders possible the detection of the measured values that can be associated with the disposable. The measured values can subsequently be used to increase the safety when using the medical examination appliance, in particular the tonometer.

In principle, the medical examination appliance can be chosen as desired, provided a disposable is usable. Typically, the medical examination appliance is embodied as an appliance that is brought into contact with the patient for the purposes of carrying out a measurement. In a particularly preferred embodiment, the medical examination appliance is a tonometer, in particular a tonometer for an applanation measurement. In this case, the disposable is a test body or a protective sleeve for a test body. Consequently, the disposable is pressed against the cornea of an eye for applanation purposes.

In variants, the disposable may also be a constituent part of a contact glass or part of a contact glass for fundoscopy. Further, the disposable can also be embodied as a protective sleeve of an otoscope.

Likewise, the detection device can have any embodiment within a wide scope and substantially depends on the type of physical property to be detected.

By way of example, the detection device may comprise an image sensor, by means of which an optical measured value of the disposable can be ascertained. By way of example, the image sensor may comprise as a CCD sensor, CMOS sensor or other sensors known to a person skilled in the art. In particular, the sensor may comprise different sensor types with possibly different recording spectra and resolutions.

However, the detection device may also comprise further sensors or means, by means of which a physical property of the disposable can be ascertained (see below in this respect).

Preferably, an operating state is assigned to the medical examination appliance, in particular the tonometer, on the basis of the measured value. Hence, a decision can be made on the basis of the measured value, in particular, as to whether or not the medical measuring appliance, in particular the tonometer, is operational. This can create a safe measuring method using the medical examination appliance. Should the examination appliance be ascertained as being non-operational on account of the measured value, a warning signal, for example, can be output. Further, as an alternative or in addition thereto, the triggering of a measurement can be electronically suppressed. Further, only a note about the operating state may also be assigned, in particular stored or printed, in the measured values.

In variants, or additionally, the measured values can be stored. By way of example, a batch number or the like can be read and can be assigned to the measurement result of the medical examination. Hence, it is possible at a later time to trace back the disposable with which a patient was examined. By way of example, thereby, patients that require a repeat medical examination can easily be ascertained in the case of a recall of a faulty batch of disposables.

Preferably, the physical property comprises the natural frequency of the disposable and/or of a part of the medical examination appliance, in particular of the tonometer, comprising the disposable.

Presently, the phrase “a and/or b” is understood to mean one of the sets {a}, {b} or {a, b}.

Here, the natural frequency serves to identify the disposable. Provided the measured natural frequency corresponds to a natural frequency associated with the disposable, the examination appliance, in particular the tonometer, can be assigned an operating state that allows a measurement to be carried out. Should a natural frequency lying outside of an admissible frequency interval be ascertained, the examination appliance can be associated with an operating state that does not allow a measurement to be carried out. As an alternative or in addition thereto, the ascertained natural frequency or a piece of information correlating therewith can be stored or output together with the measurement result of the examination appliance, in particular the applanation pressure, for example.

Here, the natural frequency need not necessarily be ascertained from the disposable as such. Instead, a plurality of constituent parts of the medical examination appliance could also be made to vibrate together with the disposable in order to ascertain a meaningful natural frequency. Here, the disposable preferably influences the natural frequency of the vibrating system sufficiently such that a missing disposable, an incorrectly assembled disposable or a non-compatible disposable can be identified.

Moreover, by generating a vibration, it can be tested whether the disposable is sufficiently securely assembled on the disposable-holding apparatus such that it cannot detach from the disposable-holding apparatus, even during the examination.

In variants, determining the natural frequency can also be dispensed with. In this case, the disposable and/or a part of the medical examination appliance comprising the disposable can be made to vibrate differently or carry out any other movement, as a result of which conclusions about the disposable can be obtained.

Preferably, for the purposes of ascertaining the natural frequency, the disposable and/or a part of the medical examination appliance comprising the disposable are/is made to vibrate by an apparatus for generating an applanation force. Hence, the medical examination appliance preferably comprises a tonometer that comprises an actuator for exerting an applanation force. This actuator can have different embodiments.

In variants, a separate apparatus can be provided, as a result of which the disposable or the disposable together with the disposable-holding apparatus can be made to vibrate.

Preferably, the vibration is generated electromagnetically. To this end, the actuator comprises an electromagnet, by means of which a force can be exerted on the disposable-holding apparatus with the disposable. The use of an electromagnet is advantageous in that only few moving parts are required; in particular, a guide for the disposable-holding apparatus and a magnetic core carrying the disposable-holding apparatus may be substantially sufficient. Naturally, the core can also be stationary in an alternative thereto and the disposable-holding apparatus can be connected to a coil that interacts with the magnetic core.

By way of example, for the purposes of ascertaining the natural frequency, can be ascertained directly on the basis of the curve of the magnetic field strength, the recorded current, the number of turns of the coil, etc. Further, the natural frequency can also be ascertained by way of a position sensor, by means of which a frequency of the disposable can be ascertained.

Alternatively, the actuator can also be provided mechanically by a stepper motor or the like, by means of which a force can be applied to the disposable-holding apparatus. Further, provision can also be made of a hydraulic or pneumatic system with pressure cylinders for exerting the applanation pressure. The actuator can also act with or counter to a spring force. Further, the actuator may also comprise damping elements for damping the movement of the disposable. A person skilled in the art knows of further possible techniques that can be used to apply a force to the disposable-holding apparatus for the purposes of exerting an applanation pressure.

Preferably, at least one of the following features is determined on the basis of the measured value of the natural frequency:
an alignment of the disposable on the medical examination appliance;
a mass of the disposable;
a mass distribution, in particular a center of mass of the disposable.

This can be used to ascertain whether the disposable, on the one hand, is an admissible disposable. This is important in order to be able to obtain the best-possible measurement results by way of the medical examination appliance. Further, this ensures that the patient is not put at risk by way of untested disposables.

Further, this can be used to ascertain whether the disposable is correctly positioned. Provided the disposable has a cap-like embodiment, this can be used to determine, for example, whether the disposable has been placed onto the disposable-holding apparatus up to the stop.

Since the disposable-holding apparatus and the vibration behavior thereof are known, it is possible to deduce, e.g., a center of mass of the disposable, etc., on account of the changes in the vibration behavior. This can be used, in turn, to ascertain whether this is an admissible and correctly positioned disposable.

It is obvious to a person skilled in the art that it is not necessarily mandatory to make a distinction between correct positioning and correct disposable. In principle, it is sufficient within the method to be able to determine whether or not the examination appliance is operational.

In variants, other features of the disposable may also be ascertained instead of the aforementioned features, for example a damping behavior or the like.

In a further preferred embodiment, the physical property comprises an optical reflection behavior of the disposable. Preferably, the optical reflection behavior is ascertained by an image sensor, in particular by an image sensor of a slit lamp. In variants, separate sensors may also be provided, which are comprised by the tonometer. In the method, light reflected by the disposable is preferably detected by the image sensor and evaluated electronically. Subsequently, the evaluated data can be compared to reference values. Provided the evaluated data lie in a predetermined reference range, the examination appliance can be assigned an operating state that allows a measurement to be carried out. On the other hand, a warning, for example, can be output or the examination appliance can be electronically blocked for examinations, for example.

In variants, or additionally, further physical properties of the disposable may also be measured, in particular directly or indirectly. Provided the disposable is at least slightly electrically conductive, it is possible, for example, to ascertain a resistance in the disposable. A person skilled in the art is aware of further options.

Preferably, the optical reflection behavior comprises an optical detection of data of the disposable. The data may relate to different information items, in particular to one or more of the following information items:
manufacturer, e.g., company, country of manufacturer, etc.;
type designation;
batch number;
production date;
expiry date;
list of tonometers admissible therefor;
etc.

The data allow ascertainment as to whether the disposable is admissible for the present tonometer, even before an examination is carried out using the examination appliance. This can be implemented by comparing a type number of the disposable with a positive list of the tonometer. Further, the disposable may have already detected the admissible tonometers in the data. Further, a decision as to whether the disposable may still be used can be made on the basis of the production date or expiry date. One or more of the information items listed above may be assigned to the measurement result of the examination appliance. To this end, the information items can be stored or output together with the measurement result, in particular the measurement result of the tonometer. A person skilled in the art is aware of further information items that can be additionally recorded on the disposable. Moreover, the information items may also be used in other ways following the detection by the detection device.

In variants, the detection of data of the disposable may also be dispensed with.

Preferably, the data of the disposable comprise a code, in particular a two-dimensional code such as, e.g., a QR code or a one-dimensional code such as, e.g., a barcode, wherein the code is detected with the medical examination appliance. Hence, a lot of information can be housed on the disposable with little space required. This is particularly advantageous since a disposable is relatively small. Other codes known to a person skilled in the art may also be used in place of the QR code or the barcode. The code can be directly convertible into the corresponding information items such as batch number, manufacturer, etc. Alternatively, the code may also merely comprise a pointer for a list, by means of which, for example, a line number of a list is referred to by a number of the code. This can further reduce the spatial requirement. In this respect, a person skilled in the art is aware of further options.

In variants, the code may also comprise letters and numbers. The code need not necessarily be encrypted but may also be present as plain text. Further coding techniques are known to a person skilled in the art.

A test body for use in a tonometer comprises a contact area for contacting the cornea and a code, which is detectable, in particular, by a detection device of the examination appliance, in particular of the tonometer.

Preferably, the contact area comprises a code, which is detectable by a detection device of the examination appliance, in particular of the tonometer.

In variants, the code may also be disposed outside of the contact area (see below).

Preferably, the code is presented centrally on the contact area, in particular on an area of less than 3×3 mm, particularly preferably less than 2×2 mm, more particularly preferably on approximately 1.5×1.5 mm. During the tonometric measurement, a pressure is typically applied to the disposable until a predetermined applanation area is reached. The form of the applanation area is ideally circular but may also be oval or differently shaped in practice. However, in virtually any case, a central region of the test body, in particular of the disposable, is within an applanation area such that information in respect of the disposable can be reproduced in this central region. The maximum area available therefore depends on the type of measurement method. By way of example, in a Goldmann-type measurement, an applanation area with a diameter of 3.06 mm is sought after. Hence, an inscribed circle square may have an edge length of 2.16 mm, for example. On account of possible deviations from the ideal circular form, a region with an edge length of less than 2 mm, in particular a region with an edge length of approximately 1.5 mm, is preferably used for the code in practice in a Goldmann-type method.

In principle, the region can be chosen to be as small as desired. The possible information content of the region in this case depends on the type of coding and on the resolution of the detection apparatus.

Naturally, the region may also have other outer contours (rectangular, round, etc.). Depending on the type of measurement method, the central region for the code may also be chosen to be greater than 3×3 mm.

In a further preferred embodiment, the code has a circular ring-shaped embodiment and is arranged in a peripheral region with respect to the contact area. Using this, the code can be disposed around the region that is relevant to the measurement and nevertheless be detectable by the detection apparatus.

Preferably, contaminations and/or damage of/to the disposable are/is ascertained on the basis of the optical reflection behavior. Even if this relates to a correctly positioned and admissible disposable, an examination can be impaired by contaminations. Now, the reflection behavior allows the determination as to whether the disposable has contaminations and an operating state can be assigned to the medical examination appliance, in particular the tonometer, on the basis of the measurement results. Further, the detection device may also determine possible damage to the disposable, as a result of which an operating state can be assigned accordingly. In the preferred embodiment, the detection device is used to detect the entire region of the disposable relevant to the measurement such that substantially all damage or contaminations to/of the disposable, which could impair a medical examination, is/are detected. This is then particularly the case if an image sensor is used as a detection device, said image sensor measuring the applanation area at the same time (see below).

As an alternative or in addition thereto, further parts of the tonometer can also be tested for integrity and contaminations. In particular, the disposable-holding apparatus can be checked for damage and/or contaminations in a light-guiding region. In variants, the detection of contaminations can also be dispensed with.

Preferably, the optical reflection behavior is ascertained with an image sensor of the examination appliance and/or a slit lamp. The image sensor or sensors is/are part of the detection device. Hence, the image sensor is preferably comprised by an examination appliance. In the preferred embodiment, the image sensor is comprised by a slit lamp and the medical examination appliance comprises both a slit lamp and a tonometer. The tonometer may also be present as an autonomous appliance which can be coupled to the slit lamp, for example when necessary, such that an image evaluation can be implemented by way of the corresponding device of the slit lamp. The tonometer may also be securely connected to the slit lamp and may be able to be pivoted into the beam path of the slit lamp, for example when necessary. A person skilled in the art is aware of further variants.

Particularly preferably, an applanation area is electronically measured by the image sensor of the slit lamp when the tonometer is used. Firstly, this can achieve a particularly precise measurement. Secondly, different examinations can be carried out therewith using the same image sensor, as a result of which, on the one hand, the apparatus and hence also the method can be cost-effective. Further, light outside of the visible light spectrum can hence be used for the tonometric examination, as a result of which a more comfortable eye examination can be achieved for the patient. Moreover, it is hence possible to carry out a tonometric measurement without fluorescein. The contact area can be detected electronically by way of a tear film depot between the disposable and the cornea.

To this end, the tonometer can be embodied in such a way that the disposable is pressed against the cornea of an eye by way of an actuator. Here, the actuator is preferably controlled on the basis of the applanation area that is ascertained by way of the image sensor. To this end, an applanation pressure generated by the actuator can be stored or output when a predetermined applanation area is reached. A person skilled in the art is aware of corresponding methods.

In variants, the detection device may also be comprised by a different examination appliance. The detection device present as a separate apparatus or can be part of the tonometer.

An apparatus for carrying out a method according to any one of claims 1 to 11 comprises a tonometer with a disposable-holding apparatus, wherein the disposable-holding apparatus comprises a ring illumination that is embodied as a stop for an edge of a cap-shaped disposable. Therewith, light can be guided by the ring illumination through the wall of the cap-shaped disposable to the contact area is a method. In the region of the applanation, the contact area becomes dark, as a result of which the applanation area can be detected and evaluated with a detection device, in particular a photosensor.

Further advantageous embodiments and feature combinations of the invention emerge from the following detailed description and the totality of the patent claims.

### BRIEF DESCRIPTION OF THE DRAWINGS

In the drawings used to explain the exemplary embodiment:

FIG. 1 shows a schematic illustration of a side view of a tonometer head;

FIG. 2 shows an illustration according to FIG. 1 with a fitted disposable;

FIG. 3 shows an illustration according to FIG. 2, wherein the disposable is presented as a sectional image;

FIG. 4 shows a schematic side view of a slit lamp comprising a tonometer;

FIG. 5 shows a schematic plan view of a disposable with a code applied centrally to the contact area; and

FIG. 6 shows a schematic plan view of a disposable with a code applied in the edge region of the contact area.

In principle, the same parts in the figures have been provided with the same reference signs.

### WAYS OF IMPLEMENTING THE INVENTION

In general, the figures explained in the following are greatly simplified. Detailed representations of optical components, in particular lens systems for magnification and focusing, filters, etc., are dispensed with deliberately since a person skilled in the art is sufficiently well aware of the structure thereof.

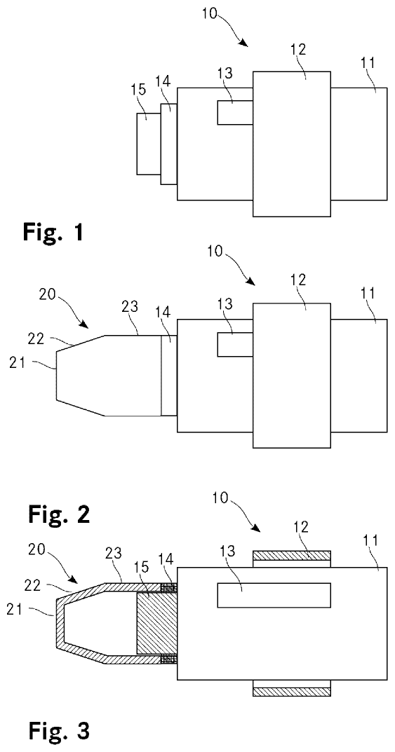

FIG. 1 shows a schematic illustration of a side view of a tonometer head 10. The tonometer head 10 comprises a tonometer body 11. The latter has a ring illumination 14 and a base 15 for receiving a test body embodied as a disposable.

The tonometer body 11 is guided in a longitudinally displaceable fashion in a guide apparatus 12. The guide apparatus 12 comprises an electrically actuatable coil for generating a magnetic field while the tonometer body 11 comprises a magnetic element not illustrated in any more detail. If a magnetic field is now generated in the coil, a force is applied to the tonometer body 11 in the guide apparatus 12 and said tonometer body is displaced in the longitudinal direction.

In order to be able to determine the position of the tonometer body 11 relative to the guide apparatus 12, the tonometer body 11 comprises a position sensor 13. Such sensors are known to a person skilled in the art.

FIG. 2 shows an illustration according to FIG. 1 with a fitted disposable 20. In the present case, the disposable 20 has a cap-shaped embodiment. For a better illustration, FIG. 3 additionally shows an illustration according to FIG. 2, where the disposable 20 and the guide apparatus 12 are presented as a sectional image.

The disposable 20 comprises a contact area 21, which is pressed against the cornea in the method for ascertaining an internal pressure of an eye. The contact area 21 is adjoined by a circular conical frustum sheath-shaped region 22, which merges into a circular cylinder sheath-shaped region 23. In the present case, this is produced from a clear plastic, in particular acrylic glass or PMMA (polymethylmethacrylate). Acrylic glass should be particularly preferred because it transmits light particularly well. However, it is evident to a person skilled in the art that other materials can also be used.

The tonometer head 11 comprises a base 15, on which the disposable 20 is fitted. The base 15 comprises an optical channel that completely passes through the tonometer head 11 in the longitudinal direction.

The disposable is placed on the base 15 of the tonometer body 11 and pushed as far as the stop. Consequently, the disposable is held in force-fit fashion. In the present case, the stop is formed by a ring illumination 14. Said ring illumination contacts the edge of the disposable 20 when the latter is assembled. Therewith, light can be guided from the ring illumination 14 to the contact area 21 through the sheath areas 23 and 22. If the contact area 21 is now pressed against a cornea of an eye, the area in contact with the cornea appears dark. The dark area can thereupon be measured in order to be able to test, for example according to Goldmann, whether the predetermined applanation area has been reached. During the application, the magnetic field of the coils in the guide apparatus 12 is actuated on the basis of the ascertained applanation area in such a way that the contact pressure of the disposable 20 on the cornea of the eye can be regulated. To this end, the applanation area is continuously measured by means of a detection device. Finally, the force that is required for applanation of an area of 7.35 mm² or a circular area with a diameter of 3.06 mm is determined. The force can be calculated on the basis of the magnetic field strength. Further, the force can also be deduced using the position sensor 13 on the basis of the longitudinal displacement of the tonometer body 11 relative to the guide apparatus 12.

Now, the tonometer head can not only be moved in linear fashion but also be made to vibrate by means of the actuator, i.e., by means of the guide apparatus 12 and the tonometer head 11. The natural frequency changes according to the mass distribution of the tonometer head 11, and so whether the tonometer 10 is in an operational state can be ascertained on the basis of the natural frequency. To this end, a bandwidth of the admissible natural frequency can be predetermined. An operational state can be assigned to the tonometer when the measured value of the natural frequency lies in the bandwidth, otherwise, a warning, for example, can be output. This can prevent the disposable 20 from not being placed correctly on the base. Further, the use of unsuitable disposables can be avoided. Not least, this can prevent a measurement from being carried out entirely without a disposable.

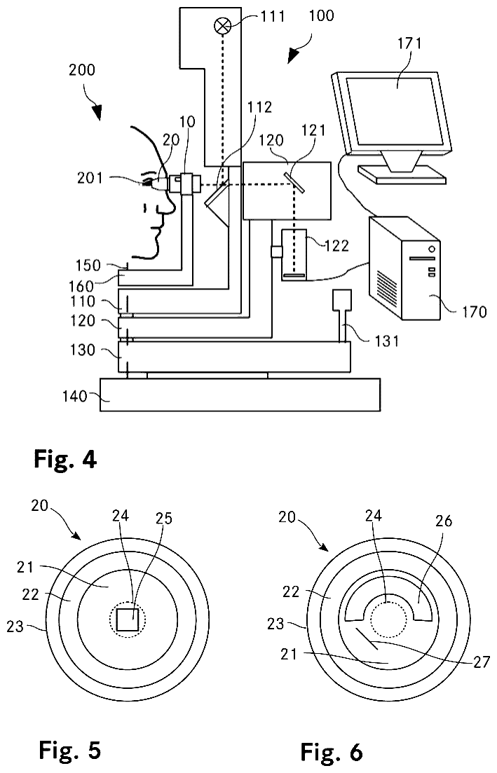

FIG. 4 shows a schematic side view of a slit lamp 100 comprising a tonometer 10.

The slit lamp 100 comprises a tonometer arm 160, an illumination unit 110 and an optics part 120, which are assembled on a cross slide 130. The cross slide 130 itself is assembled on a base plate 140, which can be embodied as a tabletop, and can be displaced in the X-, Y- and Z-direction. The slide 130 is controllable by way of an operating element 131.

The slit lamp 100 comprises a principal pivot shaft 150, by means of which the L-shaped tonometer arm 160, the illumination unit 110 and the optics part 120 are mounted in pivotable fashion. The tonometer arm 160 comprises a tonometer 10, which can be pivoted into the beam path of the slit lamp 100 when necessary.

The illumination unit comprises an illuminant 111, which can guide light to an eye 201 of a patient 200 via a semitransparent mirror 112. The optics part 120 comprises a deflection mirror 121, by means of which entering light can be guided from the eye to an image sensor 122, both during the application as a tonometer and during the application as a slit lamp. The image sensor 122, in turn, is connected to a computer, a separate computer 170 in the present case, by means of which the image data of the image sensor 122 can be evaluated. The computer 170 is connected to a monitor 171, on which the image data of the image sensor 122 can be observed. In principle, more than one image sensor may also be provided, allowing different spectra (UV, IR, etc.) to be covered, for example.

Now, the image sensor 122 can not only ascertain the applanation area but can also check a state of the disposable 20.

In particular, an image analysis, for example, can ascertain whether the contact area is clean and free from damage such that a correct measurement of the intraocular pressure can be obtained.

Within the method, at least one image is recorded using the image sensor 122 and analyzed following the assembly of the disposable 20 on the tonometer body 11 and before the tonometric measurement is carried out. In the process, it is possible, for example, to determine whether the contact area has scratches, tears, breaks, contaminations, etc. An operating state can be assigned to the tonometer on the basis of the findings. Should the damage be too great, a warning can be output, or the tonometer can be electronically blocked such that no measurements can be carried out. Alternatively, a calibration can be carried out in the case of little damage or irregularities such that the subsequent tonometric measurement can be adjusted in relation to the damage or irregularities. A normal operating state can be assigned directly if no damage or contaminations is/are ascertained, whereupon the tonometric measurement can be carried out.

Further, the image sensor 122 can also be used to ascertain whether further parts of the tonometer 10, in particular the optical parts, have contaminations or damage. By way of example, a surface of the base 15 can be checked in respect of contaminations or damage.

In a further embodiment, a test of the correct seat and the correct type of the disposable by way of generating a vibration using the coils contained in the guide apparatus 12 and the magnetic element of the tonometer body 11 and a test of the state of the contact area of the disposable are carried out successively or simultaneously. This achieves a particularly safe method for a tonometric measurement.

FIG. 5 shows a schematic plan view of a disposable 20 with a code 25 applied centrally on the contact area 21. Here, the viewing direction is directed into the opening of the disposable 20.

In practice, the contact area 21 of the disposable 20 merely comes into contact with the cornea by way of a contact region 24 that is largely known in advance. In a method according to Goldmann, the applanation area has an area of 7.35 mm² and is substantially circular. The region in the interior of this area is not of interest since only the contour of the area is measured for determining the area. Typically, one or more diameters are ascertained in order finally to calculate the area. The inner region of this area can now be used to print or engrave information. In the present case, a QR code 25 is now provided within the region, which QR code can be detected by the image sensor 122 and can be evaluated by means of a computing unit 170. The QR code comprises information, by means of which the disposable can be identified. In the present case, the information comprises the manufacturer, the production date, the expiry date, a batch number and a serial number. The serial number, in particular, can be used to ascertain whether the disposable is new, i.e., whether it has not yet been used for a measurement.

As a further variant, FIG. 6 shows a schematic plan view of a disposable 20 with a code 26 applied in the edge region of the contact area 21. The code 26 is disposed outside of the region of the contact area 21 that is required for the tonometric measurement. The code 26 has the form of a semicircular ring; however, it may also have the form of a circular ring that completely surrounds the code 26. In the present case, the code 26 is embodied as a circular barcode and comprises the same information as the above-described QR code 26. During the method, the code 26 is likewise recorded by the image sensor 122 and evaluated by the computer 170 such that the disposable 20 can be identified.

Further, the contact area 21 of the disposable 20 comprises a reference size 28 in the form of a line with a defined length. This can be used to calibrate the appliance.

The image sensor need not necessarily be provided by a slit lamp. The image sensor may also be comprised directly by the tonometer itself or by any other examination appliance.

The computing unit or the computer need not be embodied as an autonomous appliance but may be part of the slit lamp.

Instead of the electromagnetic actuator, a linear drive or other means for displacing the tonometer head, known to a person skilled in the art, may also be provided.

However, the disposable may also be produced from other transparent plastics, glass or the like. The disposable need not necessarily have a cap-shaped embodiment but can, for example, also have a cylindrical embodiment, in particular made of bulk material, etc. In this case, the tonometer body may also comprise a cylindrical depression instead of the base, it being possible to insert the disposable in said depression. The disposable need not necessarily be held in force-fit fashion; instead, it may also be held in interlocking fashion or in interlocking and force-fit fashion. To this end, a person skilled in the art knows of any number of options, such as, for example, a bayonet closure, screw closure, etc.

In conclusion, it should be noted that, according to the invention, a method for testing a disposable is developed, by means of which a medical examination method, in particular a tonometric measurement method, is able to be carried out in a safer and more robust manner.

1. Method for testing a disposable on a medical examination appliance, more particularly on a tonometer, wherein the medical examination appliance
comprises a disposable holding apparatus, on which a disposable is held and, in particular, able to be brought into contact with a patient's eye for an applanation tonometry measurement;
comprises a detection device, by means of which a physical property of the disposable can be measured;
characterized in that
a measured value of the physical property of the disposable is ascertained by means of the detection device.

2. Method according to claim 1, characterized in that an operating state is assigned to the medical examination appliance, in particular the tonometer, on the basis of the measured value.

3. Method according to claim 1, characterized in that the physical property comprises the natural frequency of the disposable and/or of a part of the medical examination appliance, in particular of the tonometer, comprising the disposable.

4. Method according to claim 3, characterized in that, for the purposes of ascertaining the natural frequency, the disposable and/or a part of the medical examination appliance, in particular of the tonometer, comprising the disposable are/is made to vibrate by an apparatus for generating an applanation force.

5. Method according to claim 3, characterized in that the vibration is generated electromagnetically.

6. Method according to claim 3, characterized in that at least one of the following features is determined on the basis of the measured value of the natural frequency:
an alignment of the disposable on the medical examination appliance;
a mass of the disposable;
a mass distribution, in particular a center of mass of the disposable.

7. Method according to claim 1, characterized in that the physical property comprises an optical reflection behavior of the disposable.

8. Method according to claim 7, characterized in that the optical reflection behavior comprises an optical detection of data of the disposable.

9. Method according to claim 8, characterized in that the data of the disposable comprise a code, in particular a QR code or a barcode, wherein the code is detected with the medical examination appliance, in particular the tonometer and/or a slit lamp.

10. Method according to claim 7, characterized in that contaminations of the disposable are ascertained on the basis of the optical reflection behavior.

11. Method according to claim 6, characterized in that the optical reflection behavior is ascertained with an image sensor of the examination appliance, in particular of the tonometer, and/or a slit lamp.

12. Test body for use in a tonometer, wherein the test body comprises a contact area for contacting the cornea, characterized in that the test body comprises a code which, in particular, is detectable by a detection device of the tonometer and/or of a slit lamp.

13. Test body according to claim 12, characterized in that the contact area comprises a code, which is detectable by a detection device of the tonometer and/or of a slit lamp.

14. Test body according to claim 12, characterized in that the code is presented centrally on the contact area, in particular on an area of less than 3×3 mm, particularly preferably less than 2×2 mm, more particularly preferably on approximately 1.5×1.5 mm.

15. Test body according to claim 12, characterized in that the code has a circular ring-shaped embodiment and is arranged in a peripheral region with respect to the contact area.

16. Apparatus for carrying out a method according to claim 1, comprising a tonometer with a disposable-holding apparatus, characterized in that the disposable-holding apparatus comprises a ring illumination that is embodied as a stop for an edge of a cap-shaped disposable.

17. Test body according to claim 13, characterized in that the code has a circular ring-shaped embodiment and is arranged in a peripheral region with respect to the contact area.
